(19) United States
(12) Patent Application Publication
(10) Pub. No.: US 2013/0209380 A1
(43) Pub. Date: Aug. 15, 2013

(54) MOISTURIZING COMPOSITION WITH SPF 30

(71) Applicants: Karine Nadau Fourcade, Villeneuve-Loubet (FR); Claire Mallard, Mougins (FR); Laurent Fredon, Roquefort Les Pins (FR)

(72) Inventors: Karine Nadau Fourcade, Villeneuve-Loubet (FR); Claire Mallard, Mougins (FR); Laurent Fredon, Roquefort Les Pins (FR)

(73) Assignee: GALDERMA S.A., Cham (CH)

(21) Appl. No.: 13/807,697

(22) PCT Filed: Jun. 29, 2011
(86) PCT No.: PCT/EP2011/060967

§ 371 Date: Mar. 22, 2013

Related U.S. Application Data
(60) Provisional application No. 61/344,335, filed on Jun. 30, 2010.

(51) Int. Cl. A61K 9/00; A61Q 17/04; A61K 8/67; A61Q 19/00; A61K 8/40; A61K 8/34; A61K 8/42; A61K 31/315; A61K 8/37

(52) U.S. Cl. CPC A61K 9/0014; A61K 31/315; A61Q 17/04; A61K 8/678; A61K 8/37; A61K 8/40; A61K 8/345; A61K 8/42; A61Q 19/007
USPC 424/59

(57) ABSTRACT

Described is a composition for topical application in the form of a moisturizing composition including zinc gluconate and at least one UVA/UVB sunscreen as a moisturizering ingredient, and the use thereof as a cosmetic or a pharmaceutical product. The composition is intended for use in the treatment of acne.

The present invention relates to compositions for topical application, and to the uses thereof as cosmetic or pharmaceutical products, said compositions being for use in the treatment of dermatological disorders, and in particular in the treatment of acne.

Acne is a common multi-factor pathology that attacks skin rich in sebaceous glands (face, shoulder area, arms and intertriginal areas). It is the most commonly occurring form of dermatosis. The following five pathogenic factors play a determining role in the formation of acne:

1. genetic predisposition;

2. overproduction of sebum (seborrhoea);

3. androgens;

4. follicular keratinization disorders (comedogenesis); and

5. bacterial colonization and inflammatory factors.

There are several forms of acne, the common factor of all being attack of the pilosebaceous follicles. Mention may be made in particular of acne conglobata, cheloid acne of the nape of the neck, acne medicamentosa, recurrent miliary acne, necrotic acne, neonatal acne, premenstrual acne, occupational acne, acne rosacea, senile acne, solar acne and common acne.

Common acne, also known as polymorphic juvenile acne, is the most common. It comprises four stages:
stage 1 corresponds to comedonic acne characterized by a large number of open and/or closed comedones and of microcysts;
stage 2, or papulopustular acne, is of mild to moderate seriousness. It is characterized by the presence of open and/or closed comedones, of microcysts, but also of red papules and pustules. It mainly affects the face and leaves few scars;
stage 3, or papulocomedonic acne, is more serious and extends to the back, the chest and the shoulders. It is accompanied by a large number of scars;
stage 4, or nodulocystic acne, is accompanied by numerous scars. It exhibits nodules and also painful voluminous crimson pustules.

The various forms of acne described above can be treated with active agents such as anti-seborrheic agents and anti-infectives, for example benzoyl peroxide (in particular the product Eclaran® sold by the company Pierre Fabre), with retinoids such as tretinoin (in particular the product Retacnyl® sold by the company Galderma) or isotretinoin (the product Roaccutane® sold by Laboratoires Roche), or else with naphthoic acid derivatives. Naphthoic acid derivatives such as, in particular, 6-[3-(1-adamantyl)-4-methoxyphenyl]-2-naphthoic acid, which is commonly called adapalene (the product Differine® sold by the company Galderma), are widely described and recognized as active ingredients that are just as effective as tretinoin for the treatment of acne.

Some Adverse Events appear with Rx products (mainly retinoids topical/oral) which produce important related AE and frequent cutaneous side effects such as Ziana: 27% subjects with related application site AE and the most important is dry skin. This shows the importance of adjunctive therapy to improve side effects of acne drugs.

Skin Care regimen recommended by dermatologists for acne treatment encompasses the following steps:

Step 1: Wash

Step 2: Medicate (Rx treatment)

Step 3: Hydrate & Protect

It is useful to have Skin Care Products which improve Acne Signs/Symptoms (Reduce oiliness of skin; reduce/not increase comedons; reduce/not increase inflammatory lesions) and/or side effects of Acne such as decrease adverse events secondary to acne treatments (reduce Dry skin; decrease erythema; reduce stinging/burning)

In one embodiment, the present invention provides a topical dermatological/pharmaceutical composition and particularly provides a moisturizing composition.

In particular, there is a need for and particularly a facial Moisturizer for dry/irritated skin preferably with a sunscreen and preferably with SPF 30.

The present invention provides a composition having the properties and advantages of protecting the skin from the sun (UVA and UVB), of long lasting moisturizing the skin, of reducing oily skin, of reducing redness and inflammation and is highly tolerated.

The present invention provides an advantageously a single composition which moisturizes the skin and protect it at the same time. Thus, it is more convenient for a subject in need of such a composition and advantageously provide a great compliance.

One embodiment of the present invention is a moisturizing composition comprising at least one moisturizer ingredient, zinc gluconate and at least one UVA/UVB sunscreen.

In a preferred embodiment, the moisturizer ingredient is selected from: glycerol, D-panthenol, Alpha tocopheryl acetate, ceramides 5 alone or in combination.

Thus in one particular embodiment, the invention provides a composition comprising:
at least one moisturizer ingredient, selected from the list of glycerol, D-panthenol, Alpha tocopheryl acetate, ceramides 5 alone or in combination;
zinc gluconate
at least one UVA/UVB sunscreen

In a preferred embodiment of invention, the sunscreen is selected from Ethyl hexyl salicylate; Ethyl hexyl cyanodiphenylacrylate; Octocrylene alone or in combination.

The composition is for topical application. Preferably, the composition is in the form of aqueous, aqueous-alcoholic or oily dispersions, dispersions of the lotion type, aqueous, anhydrous or lipophilic gels, emulsions of liquid or semi-liquid consistency of the milk type, obtained by dispersion of a fatty phase in an aqueous phase (O/W) or vice versa (W/O), or suspensions or emulsions of soft, semi-liquid or solid consistency of the cream, gel, cream-gel, foam or ointment type, or microemulsions, microcapsules, microparticles or vesicular dispersions of ionic and/or nonionic type, in the form of sprays, or else in the form of dermal devices such as patches.

A second subject of the present invention is the use a composition according to the invention, for use in the treatment and/or prevention of dermatological conditions linked to acne treatment and particularly common acne, comedonic acne, papulopustular acne, papulocomedonic acne, nodulocystic acne, acne conglobata, cheloid acne of the nape of the neck, recurrent miliary acne, necrotic acne, neonatal acne, occupational acne, acne rosacea, senile acne, solar acne and acne medicamentosa. Preferably, the preparation of a pharmaceutical composition is intended for use in preventing, inhibiting or treating common acne.

The invention also provides a method for improving and/or preventing and/or inhibiting dermatological conditions linked to acne treatment. The invention provide also a treatment process for embellishing the skin or its surface appearance, in which a composition comprising, in a physiologically acceptable medium, a retinoid, an anti-irritant and benzoyl peroxide is applied to the skin and/or its integument annexes. In a preferred embodiment, the treatment of skin is for skin with an acneic tendency or for combating the greasy appearance of the skin or the hair.

Throughout the present text, unless otherwise specified, it is understood that, when concentration ranges are given, they include the upper and lower limits of said range. Similarly, unless otherwise indicated, the proportions of the various constituents of the composition are expressed as percentage by weight (m/m) of the total weight of said composition

The composition of the invention comprise Zinc gluconate (also called zincum gluconium) is the zinc salt of gluconic acid. It is an ionic compound consisting of two moles of gluconate for each mole of zinc. Zinc gluconate is a popular form for the delivery of zinc as a dietary supplement.

The composition of the invention comprises at least one UVA/UVB sunscreen or sunblock. For this purpose, any known UVA/UVB sunscreen can be use. These latter are well known by the skilled artisan but we can cite among of them the chemical and mechanical UVA/UVB suncreens. As illustrating examples one can cite Ingredients like Mexoryl SX, Mexoryl XL, titanium dioxide, Parsol 1789 and titanium dioxide, considered alone or combined together such as those disclosed in WO91/11989.

Advantageously UVA/UVB sunscreens give to the subject in need a short term protection from sunburns, but also provide long term damage from wrinkles, sagging skin and premature aging.

The present invention comprises ceramides and preferably ceramides 5. Ceramides are sphingolipids that consists of a long-chain of amino alcohol to which a hydroxylated or non hydroxylated long chain fatty acid is linked via an amide bond.

Ceramides, the main stratum corneum (SC) polar lipids, play an important role in skin barrier function: Regulation of skin water barrier homeostasis; and/or Water-holding capacity.

In a preferred embodiment of invention, the composition comprise pseudo ceramide 5, known as N-(2-hydroxy hexadecanoyl)sphinganine, which is synthetic ceramide developed by L'Oreal and disclosed in U.S. Pat. No. 5,665,778. For this synthetic ceramide it has been demonstrated on In vitro human skin model, a good affinity and diffusion into the stratum corneum and induction synthesis of ceramides 1-2-3.

The composition may also comprise 18β-Glycyrrhetinic acid which is the active component in licorice root. Recent study has shown that 18β-Glycyrrhetinic acid exhibits many pharmacological activities.

The composition of the invention further comprises a preservative. As preservative, it can be mentioned among the preservatives, mention may be made, by way of non-limiting examples, of benzoic acid and its derivatives such as benzyl alcohol, also benzalkonium chloride, sodium benzoate, bronopol, chlorhexidine, chlorocresol and its derivatives, ethyl alcohol, phenethyl alcohol, phenoxyethanol, potassium sorbate, diazolidinylurea, taken alone or as mixtures.

By way of preferred preservative, mention may be made of phenoxyethanol, potassium sorbate or benzalkonium chloride, taken alone or as a mixture.

Another embodiment of invention relates to the use of a moisturizing composition as describe herein for protecting the skin from the sun (UVA and UVB), for long lasting moisturizing the skin, for reducing oily skin, for reducing redness and inflammation.

Invention also provides a non-therapeutic cosmetic treatment process for embellishing the skin or its surface appearance, in which a moisturizing composition as described above is applied to the skin and/or its integument annexes.

The present invention will now be illustrated by means of the following examples, which cannot limit the scope of the present invention.

### EXAMPLES

### Example 1

### Emulsion Formulation with Following Ingredients

Ethyl hexyl salicylate (UVB): 5%

Ethyl hexyl cyanodiphenylacrylate (UVB): 7%

Octocrylene (UVA): 3%

Zinc gluconate (0.2%)

silica (2%)+corn starch (2%)

PMMA spherica (2%)

glycerol (5%)

D-panthenol (0.2%)

Alpha tocopheryl acetate (0.5%)

ceramides 5

allantoin

beta-glycyrrhetinic acid (enoxolone)

### Example 2

### Tolerance and Acnegenicity Evaluation

This was a single center study evaluating the tolerance (Dermatological) and acnegenicity potential of a face moisturizer following six (6) consecutive weeks of test article use by a panel of approximately fifty (50) healthy, adult male and female acne-prone volunteers.

Prior to test article distribution, subjects were queried as to any subjective irritation (itching, burning and stinging) they may have been experiencing at that moment. Subjects were then evaluated by a Dermatologist who performed baseline dermatological evaluations of the face for the presence of erythema, dryness and oedema. Additionally, the following procedures were performed by Dermatologist:

Grading of the face for the presence of the following Acne Lesions:
Non-inflammatory Lesions
   Open comedones
   Closed comedones
Inflammatory Lesions
   Papules
   Pustules

Assessment of the Global Severity of Acne

Following the evaluations of acne lesions, subjects were provided with the test article and instructed to use the test article at least twice daily for the next six consecutive weeks and to record the times of test article application, as well as any comments and/or sensations observed, on the daily diary form provided to them. Additionally, subjects were instructed to call the testing facility as necessary and/or after twenty one (21) days of test article use to report any problems that might have occurred. This information was recorded on the subjects' call-in data sheets.

Following six (6) consecutive weeks of test article use the subjects returned to the testing facility and underwent the same dermatological evaluations (assessment of erythema, dryness and edema) as performed at the baseline visit. The dermatologist also performed visual evaluations for determining the total number of acne lesions, and global severity of acne, on the subjects' faces. Subjects were queried as to any subjective irritation (burning, itching or stinging) they may have experienced during the course of the study. Additionally, each subject filled out a questionnaire. As an indication of compliance, diaries and test articles were collected at the 6 week visit and the test articles weighed.

Diaries were reviewed and an adverse event form was completed for those subjects who reported safety related problems. Following Week 6 activities, subjects were dismissed from the study.

Study Procedures

Screening and Consenting Process

Prior to arrival at the testing facility each subject was screened to ensure he/she met all of the inclusion and none of the exclusion requirements. Following the screening process, subjects arrived at the testing facility and underwent the informed consent process and completed a brief medical history form.

Treatment

Baseline Visit (Day 1)

Subjective Irritation Assessment Subjects meeting all of the inclusion and none of the exclusion criteria were queried by the testing facility staff for any subjective irritation they might have been experiencing. The subjects were asked to assess the degree of the following sensations on their face that they were experiencing at their baseline visit using the scales below:

Itching

0—None No itching

1—Mild Slight itching, not really bothersome

2—Moderate Definite itching that is somewhat bothersome

3—Severe Intense itching that may interrupt daily activities and/or sleep

Burning

0—None No burning

1—Mild Slight burning sensation; not really bothersome

2—Moderate Definite warm, burning sensation that is somewhat bothersome

3—Severe Hot burning sensation that causes definite discomfort and may interrupt daily activities and/or sleep

Stinging

0—None No stinging

1—Mild Slight stinging sensation, not really bothersome

2—Moderate Definite stinging sensation that is somewhat bothersome

3—Severe Stinging sensation that causes definite discomfort and may interrupt daily activities and/or sleep

All data were recorded in the subject's data forms. Visual Evaluations of Cutaneous Tolerance following the subjective irritation query, the subjects had their face examined by a Dermatologist. The dermatological evaluations included erythema, dryness and edema grading of the face using the following scales:

Erythema Evaluation Score

| | |
|---|---|
| No observable erythema | 0 |
| Slight erythema, spotty or diffuse | 1 |
| Moderate erythema | 2 |
| Intense erythema | 3 |
| Fiery red with edema | 4 |

Edema Evaluation Score

| | |
|---|---|
| None | 0 |
| Slight | 1 |
| Moderate | 2 |
| Intense | 3 |

Dryness Evaluation Score

| | |
|---|---|
| No observable scaling | 0 |
| Fine flakes | 1 |
| Moderate flakes/scaling | 2 |
| Large flakes/severe scaling | 3 |

All findings were recorded on subjects' individual data recording forms. Visual Evaluations for Acne Lesions and Determining Global Severity of Acne following the cutaneous tolerance evaluations, the dermatologist documented on the subjects' data sheets, the number of acne lesions present on their faces. The lesion counts were taken from the facial area [forehead, left and right cheeks and chin above the jaw line (excluding the nose)]. The counts were added together to form three groups of lesion counts: inflammatory, non-inflammatory and total lesion counts. Open and closed comedones made up the non-inflammatory group; papules and pustules made up the inflammatory group and all of the lesions composed the total lesion count group.

The following are definitions of each lesion type counted:
Open comedone—A mass of sebaceous material that is impacted behind an open follicular orifice (blackhead)
Closed comedone—A mass of sebaceous material that is impacted behind a closed follicular orifice (whitehead)
Papule—A small, palpable, solid elevation less than 1 cm in diameter
Pustule—A small, circumscribed elevation of the skin which contains yellow/yellowish-white exudates.

In addition to lesion counting, the clinical evaluator assessed the overall (global) severity of each subject's acne according to the following scale:

Grade Description

0 Clear Normal, clear skin with no evidence of acne.
1 Very Mild, Skin almost clear, rare non-inflammatory and inflammatory
lesions present (less than 4 lesions total).
2 Mild, Some non-inflammatory lesions present, with few inflammatory
lesions. Less than half the face involved.
3 Mildly Moderate, Non-inflammatory lesions predominate, with multiple
inflammatory lesions present. Several to many comedones and papules/
pustules. More than half the face involved.
4 Moderate, Inflammatory lesions are more apparent. Many comedones
and papules/pustules are present. The entire face is involved.
5 Severe, Highly inflammatory lesions predominate. Variable number of
comedones with many papules/pustules.

Subject Instructions

Following the evaluations of acne lesions, each subject was given individually coded test articles (each test article was weighed prior to distribution) and instructed to use the test articles for six (6) consecutive weeks with Use Instructions to Apply to their face to clean skin at least twice a day. Applications must be at least 4 hours apart.

Mid-Study Call-In (Week 3)

The subjects were instructed to call the testing facility as necessary and/or after twenty-one (21) days of test article use to report any problems that might have occurred. During the mid-study call-in, the subjects were asked several questions.

Week 6 Visit (Day 42)

Following six consecutive weeks of at least twice daily test article use the subjects returned to the testing facility and underwent the same evaluations (subjective tolerance, cutaneous assessments, acne lesion assessments and determination of global severity of acne) as previously performed at baseline. The Medical Investigator did not have access to the subjects' previous data.

Additionally, the subjects' diary forms were collected and reviewed and for those test subjects who reported safety-related problems (e.g., dryness, burning), an adverse event form was completed. Also, as an indication of compliance, weights of the test articles were determined at the final (6-week) visit.

Finally, the subject completed a provided questionnaire.

Assignment of Treatment

Each subject who signed an informed consent form and successfully completed the screening procedures was enrolled in the study. Upon enrollment, each subject was assigned a unique subject number. Of the 59 subjects enrolled, 57 received the investigational product.

Results:

Summary of Demographics:

TABLE 1

| | | Enrolled | | Completed | |
|---|---|---|---|---|---|
| | | N = 59 | | N = 55 | |
| Age of Test | Mean | 27.2 | | 27.6 | |
| Subjects (years) | SD | 4.7 | | 4.5 | |
| | Median | 27.0 | | 27.0 | |
| | Range | 16-35 | | 16-35 | |
| Gender of Test | Female | 40 | (67.8%) | 37 | (67.3%) |
| Subjects | Male | 19 | (32.2%) | 18 | (32.7%) |
| Ethnicity | Caucasian | 32 | (54.2%) | 31 | (56.4%) |
| | Hispanic | 20 | (33.9%) | 17 | (30.9%) |
| | Asian/Pacific Islander | 6 | (10.2%) | 6 | (10.9%) |
| | Other | 1 | (1.7%) | 1 | (1.8%) |
| Fitzpatrick Skin | I | 4 | (6.8%) | 4 | (7.3%) |
| Type | II | 8 | (13.6%) | 7 | (12.7%) |
| | III | 30 | (50.8%) | 26 | (50.9%) |
| | IV | 17 | (26.8%) | 16 | (29.1%) |

Summary of Serious Adverse Events

No serious adverse events were reported during this study.

Assessment of Tolerance

A summary of the data obtained from the dermatologist's evaluations of the face are located in Text Table 7-2. The scale used by the dermatologist is explained earlier.

Dermatologist's Observations of the Face:

TABLE 2
Frequency of Scores

| | Baseline | | | Week 6 | | |
|---|---|---|---|---|---|---|
| | Erythema | Edema | Dryness | Erythema | Edema | Dryness |
| | Face | | | | | |
| 0 | 52 | 55 | 55 | 39 | 55 | 54 |
| 1 | 3 | 0 | 0 | 15 | 0 | 1 |
| 2 | 0 | 0 | 0 | 1 | 0 | 0 |
| 3 | 0 | 0 | 0 | 0 | 0 | 0 |
| 4 | 0 | | | 0 | | |
| Total | 55 | 55 | 55 | 55 | 55 | 55 |
| | Dermatologist's Observations of the Face | | | | | |
| Mean | 0.05 | 0.00 | 0.00 | 0.31 | 0.00 | 0.02 |
| SD | 0.23 | 0.00 | 0.00 | 0.50 | 0.00 | 0.13 |
| Median | 0.00 | 0.00 | 0.00 | 0.00 | 0.00 | 0.00 |
| N | 55 | 55 | 55 | 55 | 55 | 55 |

The data in Text Table 2 reveal the following:
A significant increase, relative to baseline, in erythema values was observed by the dermatologist on the subjects' faces after 6 weeks of test article use; and
No significant change, relative to baseline, in dryness or edema values was observed by the dermatologist on the subjects' faces after 6 weeks of test article use.

The data obtained from the subjective tolerance questionnaire are located in Text Table 3.

Subjective Tolerance:

TABLE 3

| | Frequency of Scores | | | | | | | |
|---|---|---|---|---|---|---|---|---|
| | Itching | | Burning | | Stinging | | Other Sensations | |
| | Baseline | Week 6 | Baseline | Week 6 | Baseline | Week 6 | Baseline | Week 6 |
| 0 | 55 | 52 | 55 | 48 | 55 | 51 | None | None |
| 1 | 0 | 3 | 0 | 7 | 0 | 4 | Headache | Tingling, Redness, |
| | | | | | | | | Warming, Dryness |
| 2 | 0 | 0 | 0 | 0 | 0 | 0 | None | None |
| 3 | 0 | 0 | 0 | 0 | 0 | 0 | None | Breakout and |
| | | | | | | | | Cooling |
| Total | 55 | 55 | 55 | 55 | 55 | 55 | 1 | 8 |

| Subjective Tolerance | | |
|---|---|---|
| Itching | Burning | Stinging |

| | Baseline | Week 6 | Baseline | Week 6 | Baseline | Week 6 |
|---|---|---|---|---|---|---|
| Mean | 0.00 | 0.05 | 0.00 | 0.13 | 0.00 | 0.07 |
| SD | 0.00 | 0.23 | 0.00 | 0.34 | 0.00 | 0.26 |
| Median | 000 | 0.00 | 0.00 | 0.00 | 0.00 | 0.00 |
| N | 55 | 55 | 55 | 55 | 55 | 55 |

The data in Text Table 3 reveal the following:
A significant increase, relative to baseline, in the degree of burning reported by the subjects after 6 weeks of test article use; and
No significant change, relative to baseline, in the degree of itching or stinging, reported by the subjects after 6 weeks of test article use.

Acnegenicity Potential

A summary of baseline and post-baseline values for the acne lesion counts are shown in Text Table 4:

| | Descriptive | | |
|---|---|---|---|
| Parameter | Statistics | Baseline | Week 6 |
| GLOBAL | Mean | 2.05 | 1.95 |
| SEVERITY | SD | 0.68 | 0.73 |
| | Median | 2.00 | 2.00 |
| | N | 55 | 55 |
| | Percent Change* | | −5.31% |
| OPEN | Mean | 5.24 | 2.07 |
| COMEDONES | SD | 7.67 | 4.26 |
| | Median | 2.00 | 0.00 |
| | N | 55 | 55 |
| | Percent Change* | | −60.42% |
| CLOSED | Mean | 12.75 | 6.45 |
| COMEDONES | SD | 8.46 | 5.99 |
| | Median | 10.00 | 5.00 |
| | N | 55 | 55 |
| | Percent Change* | | −49.36% |
| PAPULES | Mean | 3.82 | 4.09 |
| | SD | 3.92 | 5.93 |
| | Median | 2.00 | 3.00 |
| | N | 55 | 55 |
| | Percent Change* | | 7.14% |
| PUSTULES | Mean | 0.05 | 0.00 |
| | SD | 0.30 | 0.00 |
| | Median | 0.00 | 0.00 |
| | N | 55 | 55 |
| | Percent Change* | | −100.00% |
| TOTAL | Mean | 21.85 | 12.62 |
| LESION | SD | 12.98 | 9.93 |
| COUNT | Median | 17.00 | 11.00 |
| | N | 55 | 55 |
| | Percent Change* | | −42.26% |

*Intra-Subject Change = Difference in values at two different time points
**P-Values generated from a Wilcoxon Signed-Rank Test. Bolded values are significant at P < 0.05.

The results indicate that when the changes in post-baseline values for the number of acne lesions present on the subjects' faces were compared to baseline values:
The dermatologist did not observe a significant change, relative to baseline, in the overall global severity of acne present on the subjects' faces after 6 weeks of test article use;
The dermatologist observed a significant decrease (improvement), relative to baseline, in the amount of open comedones, closed comedones and total lesion count present on the subjects' faces after 6 weeks of test article use; and
The expert evaluator did not observe a significant change, relative to baseline, in the amount of papules or pustules present on the subjects' faces after 6 weeks of test article use. Since no significant increase in total lesion counts was detected, the claim “non-acnegenic” can be supported for test article Cetaphil Acne Moisturizer SPF 30.

Assessment of Cosmetic Acceptability

A Sponsor provided questionnaire was administered to each subject after 6 weeks of daily test article use. The questionnaire was designed to gauge the subjects' opinions of the test articles. A summary of the questionnaire responses are displayed in Text Table 5.

Text Table 5 Questionnaire Responses

| | | For each of the characteristics below, please tell us whether you | | | | | | |
|---|---|---|---|---|---|---|---|---|
| | | agree or disagree: The product . . . | | | | | | |
| | What is | | | | | | Does not | |
| | your overall | | | Makes | Does not | | leave a | Hydration |
| | impression | Reduces | Reduces | skin soft | make | | white | seems to |
| | of this | skin | skin | and | the skin | Is non- | residue on | last a long |
| | product? | roughness | dryness | smooth | sticky | greasy | the skin | time |
| Successes | 51 | 44 | 44 | 50 | 47 | 42 | 48 | 46 |
| Failures | 4 | 11 | 11 | 5 | 8 | 13 | 7 | 9 |
| Total | 55 | 55 | 55 | 55 | 55 | 55 | 55 | 55 |
| P-Value\hat{ } | <0.0001 | <0.0001 | <0.0001 | <0.0001 | <0.0001 | <0.0001 | <0.0001 | <0.0001 |
| Not applicable | 0 | 0 | 0 | 0 | 0 | 0 | 0 | 0 |

| | For each of the characteristics below, please tell us whether you agree or disagree: The product . . . | | | | | | | |
|---|---|---|---|---|---|---|---|---|
| | | | | Leaves | Seems | | | |
| | | | | skin | compatible | | | |
| | | | | feeling | with | | | Provides |
| | | Does not | Improves | hydrated | makeup | | | a comfort |
| | Is non- | sting or | skin | and | (Females | Does not | Is easy to | sensation |
| | irritating | burn | texture | protected | only) | clog pores | apply | to the skin |
| Successes | 49 | 47 | 43 | 48 | 35 | 52 | 55 | 47 |
| Failures | 6 | 8 | 12 | 7 | 2 | 3 | 0 | 8 |
| Total | 55 | 55 | 55 | 55 | 37 | 55 | 55 | 55 |
| P-Value\hat{ } | <0.0001 | <0.0001 | <0.0001 | <0.0001 | <0.0001 | <0.0001 | <0.0001 | <0.0001 |
| Not applicable | 0 | 0 | 0 | 0 | 18 | 0 | 0 | 0 |

| | For each of the characteristics below, please tell us | | | | | | | |
|---|---|---|---|---|---|---|---|---|
| | whether you agree or disagree: The product . . . | | | | | | | |
| | | | Fits the | Is easy to | | | | |
| | Helps the | | specific | incorporate | | | | |
| | skin to | Helps | needs | it in my | How would you rate the following | | | |
| | appear less | reduce oily | of my | daily | characteristics of the product? | | | |
| | greasy/oily | skin feeling | skin | regimen | Texture | Color | Scent/Smell | Spreadability |
| Successes | 40 | 35 | 39 | 53 | 54 | 53 | 45 | 54 |
| Failures | 15 | 20 | 16 | 2 | 1 | 2 | 10 | 1 |
| Total | 55 | 55 | 55 | 55 | 55 | 55 | 55 | 55 |
| P-Value\hat{ } | 0.0005 | 0.0290 | 0.0013 | <0.0001 | <0.0001 | <0.0001 | <0.0001 | <0.0001 |
| Not applicable | 0 | 0 | 0 | 0 | 0 | 0 | 0 | 0 |

| | How would | How would | What characteristics best describe the | | |
|---|---|---|---|---|---|
| | you rate the | you rate the | product? | | |
| | product's | product's | During | Just after | During |
| | consistency? | absorption? | application | application | the day |
| Successes | 46 | 44 | 55 | 50 | 51 |
| Failures | 9 | 11 | 0 | 5 | 4 |
| Total | 55 | 55 | 55 | 55 | 55 |
| P-Value\hat{ } | <0.0001 | <0.0001 | <0.0001 | <0.0001 | <0.0001 |
| Not applicable | 0 | 0 | 0 | 0 | 0 |

| | Between the | | | |
|---|---|---|---|---|
| | test product | | How likely | |
| | and your | | are you to | |
| | current facial | | recommend | |
| | moisturizer (if | Would you | this | |
| | applicable), | consider | moisturizer | |
| | which product | switching to | to your | Would you |
| | do you | the test | family and | buy this |
| | prefer? | product? | friends? | product? |
| Successes | 17 | 28 | 47 | 32 |
| Failures | 26 | 27 | 8 | 23 |
| Total | 43 | 55 | 55 | 55 |
| P-Value\hat{ } | 0.9369 | 0.5000 | <0.0001 | 0.1403 |
| Not applicable | 12 | 0 | 0 | 0 |

\hat{ }P-Value determined using an exact binomial test. Bolded values are significant at P ≦ 0.05.

The significance of the questionnaire responses was determined using a binomial test with an a priori 50/50 distribution assumption. The significance of the questionnaire responses was determined using a binomial test with an a priori 50/50 distribution assumption. The responses were pooled into two categories: the positive responses (very pleasant, pleasant, strongly agree, agree, just right, very quick, quick, test product, yes, would strongly recommend and recommend) were pooled into one category (success); the negative responses (very unpleasant, unpleasant, strongly disagree, disagree, too thick, too runny, very slow, slow, other product, no difference, no opinion, no and would not recommend) were pooled into another category (failure). “Not applicable” responses were not included in the calculations.

The data from Text Table 5 show that, 6 weeks after daily test article use, there was a significant proportion (P0.05) of the population (compared to an assumed 50/50 distribution) who:
Had an overall pleasant impression of the product;
Felt the product reduced skin roughness;
Felt the product reduced skin dryness;
Felt the product made their skin soft and smooth;
Felt the product did not make their skin sticky;
Felt the product was non-greasy;
Felt the product did not leave a white residue on the skin;
Felt the hydration seemed to last a long time;
Felt the product is non-irritating;
Felt the product does not sting or burn;
Felt the product improved skin texture;
Felt the product left the skin feeling hydrated and protected;
Felt the product seemed to be compatible with makeup (Females only);
Felt the product did not clog pores;
Felt the product was easy to apply;
Felt the product provided a comforting sensation to the skin;

Additionally, there was no significant difference, from an assumed 50/50 distribution, between:
Those subjects who preferred the test product to those who preferred their regular face moisturizer or felt there was no difference;
Those subjects who would switch to the test product and those who would not switch or did not have an opinion; and
Those subjects who would buy the test product and those who would not buy the product or did not have an opinion.

Discussion and Overall Conclusions

Tolerance

Dermatologist's Evaluations

Under the conditions of this study, the Board Certified Dermatologist observed the following:
A significant increase, relative to baseline, in erythema values present on the subjects' faces after 6 weeks of test article use; and
No significant change, relative to baseline, in dryness or edema values present on the subjects' faces after 6 weeks of test article use.

Subjective Irritation Query

Based on the Subjective Irritation Questionnaire distributed to the subjects at baseline and the Week 6 visit, the subjects reported the following:
A significant increase, relative to baseline, in the occurrence of burning on the subjects' faces after 6 weeks of test article use; and
No significant change, relative to baseline, in occurrence of itching or stinging on the subjects' faces after 6 weeks of test article use.

Acnegenicity

An expert evaluator observed each subject's face to determine the following:
Global Severity
Presence of Non-Inflammatory Lesions
Open Comedones
Closed Comedones
Presence of Inflammatory Lesions
Papules
Pustules
Total Lesion Count

The data reveals the following:
The dermatologist did not observe a significant change, relative to baseline, in the overall global severity of acne present on the subjects' faces after 6 weeks of test article use;
The dermatologist observed a significant decrease (improvement), relative to baseline, in the amount of open comedones, closed comedones and total lesion count present on the subjects' faces after 6 weeks of test article use; and
The expert evaluator did not observe a significant change, relative to baseline, in the amount of papules or pustules present on the subjects' faces after 6 weeks of test article use. Since no significant increase in total lesion counts was detected, the claim “non-acnegenic” can be supported for test article Cetaphil Acne Moisturizer SPF 30.

As a conclusion, this example shows that the Non- acnegenicity of the composition which was the Main Objective of the study. With regards to the Secondary objective, the present composition according to the invention provides good tolerance. Manifestations of skin discomfort were mild in intensity and transient (possible skin discomfort when applied after shaving). With regards to the Cosmetic acceptability, the present composition according to the invention provide Good feed back.

1. A moisturizing composition comprising at least one moisturizer ingredient, zinc gluconate and at least one UVA/UVB sunscreen.

2. The moisturizing composition as defined by claim 1, wherein the composition comprises a moisturizing ingredient selected from the group consisting of: a glycerol, a D-panthenol, an Alpha tocopheryl acetate, a ceramide 5 and combinations thereof.

3. The moisturizing composition as defined by claim 1, wherein the sunscreen is selected from the group consisting of: an Ethyl hexyl salicylate; an Ethyl hexyl cyanodiphenylacrylate; an Octocrylene and combinations thereof.

4. The moisturizing composition as defined by claim 1, wherein the composition comprises a moisturizing ingredient selected from the group consisting of: a glycerol, a D-panthenol, an Alpha tocopheryl acetate, a ceramide 5 and combinations thereof;
a zinc gluconate; and
at least one UVA/UVB sunscreen selected from the group consisting of an Ethyl hexyl salicylate; an Ethyl hexyl cyanodiphenylacrylate; an Octocrylene and combinations thereof.

5. A method of treating a dermatological disorder related to differentiation and/or cell proliferation and/or keratinization; the method comprising administering to an individual subject in need thereof a moisturizing composition comprising at least one moisturizing ingredient, a zinc gluconate and at least one UVA/UVB sunscreen.

6. The method as defined by claim 5, wherein the dermatological disorder is common acne or acne vulgaris.

7. A method of treating acne-prone skin, enhancing, re-growing hair or inhibiting hair loss, fighting against skin or hair oiliness, protecting against harmful aspects of sunlight or photoinduced or chronological aging, the method comprising administering to an individual subject in need thereof an effective amount of a composition comprising at least one moisturizing ingredient, zinc gluconate and at least one UVA/UVB sunscreen.

8. A method of treating or improving acne in an individual subject in need thereof, the method comprising by administering to the individual subject a composition comprising at least one moisturizing ingredient, gluconate and at least one UVA/UVB sunscreen.
